(19) United States
(12) Patent Application Publication
(10) Pub. No.: US 2006/0282032 A1
(43) Pub. Date: Dec. 14, 2006

(54) Apparatus and method for providing lumbar support

(76) Inventors: Esther S. Smith, (US); Jeffrey Smith, (US)

Correspondence Address: Todd E. Zenger;KIRTON & McCONKIE, 1800 Eagle Gate Tower, 60 East South Temple, Salt Lake City, UT, 84111, US

(21) Appl. No.: 11/148,649

(22) Filed: Jun. 9, 2005

(51) Int. Cl. A61F 5/00

(52) U.S. Cl. 602/19

(57) ABSTRACT

An apparatus and method for providing support to the lumbar region of the torso including a support belt with an anchor above and below the belt. The anchor may be a garment coupled to the belt to improve the fashion, security and comfort of the support.

### BACKGROUND

1. Field of the Invention

The present invention relates to an apparatus and method for providing lateral support for the lumbar region of the abdomen. More specifically, the present invention provides an apparatus and method for securing a lumbar support in the lumbar region of the abdomen, and especially the back and spine.

2. Background of the Invention and Related Art

Almost all physical activity, and especially heavy lifting, places stress on the back, particularly the spine. The lumbar region of the spine, specifically the area between the sacrum and the diaphragm is susceptible to injury caused by the heavy lifting. The Center for Disease Control (CDC) estimates most of the thirteen billion dollars lost each year to muscular-skeletal injury are back injuries. In addition, to being very expensive, back injuries are extremely painful inasmuch as almost all movement places stress on the spine.

In efforts to minimize the stress on a worker's back caused by heavy lifting, many workers use back braces to provide lateral support for the lumbar region of the spine. In addition, many employers require their employees to utilize back braces to minimize the medical costs as well as productivity losses associated with back injuries. While traditional back braces have reduced the number of back injuries, there are instances where no improvement, and even some increased injuries have occurred as a result of back brace malfunctioning.

Traditional designs of back braces utilize a plurality of ridges or stays positioned vertically in line with or parallel to the user's spine, and are wrapped about the user's midsection using some type of belt-like device. While this design initially functions appropriately, over time the belt typically slides up the user's body, and thus leave the most vulnerable sections of the users spine subject to injury. This is especially true where the user of the brace becomes accustomed to the additional support.

Similarly, traditional braces are not fully adjustable. A wearer of a brace may experience discomfort, or have specific positioning needs which cannot be met by a traditional brace. For example, where the user of a brace has a particularly wide girth, and as a result the brace continually slides up the user's torso, thus exposing the lumbar region to injury, and the wearer to discomfort.

Some braces known in the prior art have a supportive belt with suspenders or shoulder straps attached thereto. Such a configuration is helpful for preventing the brace from falling down, but does not secure the brace in place.

Similarly, where a brace is not fully adjustable, the positioning of the brace is prone to move with the wearer's actions. As a result, the wearer is forced to continually tug at the brace to reposition it, thus reducing his productivity as well as simultaneously increasing his risk of injury.

### SUMMARY

In light of the above-mentioned shortcomings of the prior art, exemplary embodiments of the present invention teach an apparatus and method for providing lumbar support with anchor points both above and below to the support.

It is an object of the present invention to provide a support that is stabilized at a specific position selected by the wearer.

It is a further object of the present invention to provide a fully adjustable support to increase the comfort of the wearer.

It is a further object of the present invention to provide a plurality of straps to stabilize the support vertically.

It is a further object of the present invention to hold the support in its proper position.

### BRIEF DESCRIPTION OF THE DRAWINGS

In order that the manner in which the above recited and other features and advantages of the present invention are obtained, a more particular description of the invention will be rendered by reference to specific embodiments thereof, which are illustrated in the appended drawings. Understanding that the drawings depict only typical embodiments of the present invention and are not, therefore, to be considered as limiting the scope of the invention, the present invention will be described and explained with additional specificity and detail through the use of the accompanying drawings in which:

FIG. 1 illustrates an exemplary embodiment of the present invention.

FIG. 2 illustrates an alternative exemplary embodiment of the present invention utilizing a garment.

FIG. 3 illustrates an alternative exemplary embodiment of the present invention wherein the support is coupled to a garment, and does not utilize straps below the support.

FIG. 4 illustrates an exemplary method for using the present invention.

### DETAILED DESCRIPTION OF THE PREFERRED EMBODIMENTS

It will be readily understood that the components of the present invention, as generally described and illustrated in the figures herein, could be arranged and designed in a wide variety of different configurations. Thus, the following more detailed description of the embodiments of the system and method of the present invention is not intended to limit the scope of the invention, as claimed, but is merely representative of the presently preferred embodiments of the invention.

The term lumbar as used herein describes the abdominal segment of the torso between the diaphragm and the sacrum. Specifically, the lumbar region of the spine defined as L1-L5, and the muscles in the abdomen and back throughout that region.

The term above is defined as superior on the wearer with respect to the belt. The term below is defined as inferior on the wearer with respect to the belt.

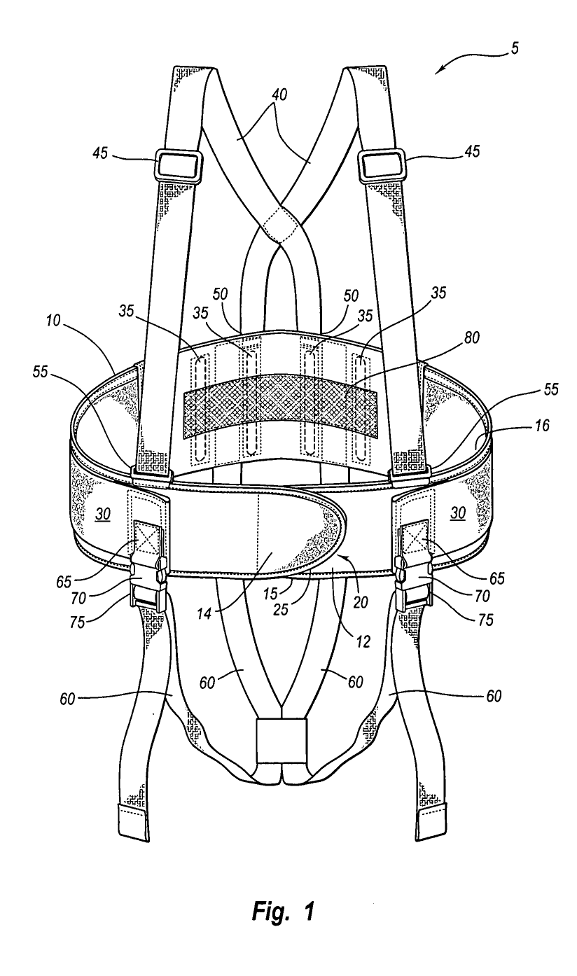

Referring now to FIG. 1, which depicts a lumbar support 5, having a first adjustable compression belt 10, having a first end 12 and a second end 14. First belt 10 further comprises a first coupling surface 15 on the inside surface on the first end 12 of the belt 10, and a second coupling surface 20 on the outside surface of the first end of the compression belt 10. The compression belt 10 also has a third coupling surface 25 on the inside of the second end 14 of the compression belt 10. First belt 10 is wrapped around the lumbar region of the wearer, placing the first end 12 of the compression belt 10 in contact with the ventral lumbar region of the abdomen and then placing the second end 14 of the compression belt in contact with first end 12, and thus forming a selectively releasable coupling link between second coupling surface 20 and third coupling surface 25. The coupleable surface as taught by the present invention may comprise a hook-and-loop surface, a zipper, a button, laces, hook & eye, snaps, or any other securing means commonly known in the art.

First belt 10, as taught by the present invention, includes providing an adjustment in the horizontal direction. Thus, the same belt may be used for a number of people with different waist sizes. In addition, the first adjustable compression belt 10 may be made of any flexible material, but as taught herein, is constructed of an elastic material to permit the wearer the maximum amount of flexibility for breathing and movement, while still holding the support in place.

Exterior to the first belt 10 is second compression adjustable belt 30, which is comprised of a belt that partially circumnavigates the lumbar region of the wearer. As taught by the present invention, the second adjustable compression belt 30 is wrapped around the back of the wearer from one hip to the other hip to provide additional compression for the support at the back of the wearer. By employing the second adjustable compression belt 30, the wearer increases the adjustability of the present invention and simultaneously improves the comfort to the wearer by providing greater flexibility increasing the support of the belt without reducing the ability of the wearer to breath or move normally. The second adjustable compression belt 30 utilizes similar coupling surfaces to those used with the first adjustable compression belt 10. Specifically, on the interior surface of the second adjustable compression belt 30 is a hook surface which will mate with the external surface 100 of the first adjustable compression belt 10, or vice versa.

On the dorsal surface of the first adjustable compression belt 10, is placed a plurality of semi-rigid stays 35, which provide additional support to the wearer's lumbar region. The stays are positioned at intervals across the back of the wearer. An alternative embodiment of the present invention teaches that the position of the stays may be adjusted depending on the anatomy of the wearer. The second adjustable compression belt 30 is positioned outside the semi-rigid stays 35, and thus improves the support provided by the stays to the lumbar region of the wearer, including the lumbar region of the spine as well as other areas of the abdomen.

Upper harness or shoulder harness 40 extends from or near the upper ridge 16 of the first belt 10 to form a suspender-like harness to prevent the harness from sliding down below the lumbar region of the wearer. The shoulder harness 40 may be made of elastic material, or any other flexible material desired, and forms a first vertical movement anchor to belt 10. To increase the comfort of the harness to the wearer, the present invention teaches providing shoulder harness adjustment slide 45 on each strap of the shoulder harness 40, or alternatively at the rear of the support, for adjustment to suit the size of the user. The first vertical movement anchor prevents belt 10 from moving vertically downward on the user, as known in the prior art.

Harness 40 connects to belt 10 using insertion points 50 positioned both dorsally and ventrally. While FIG. 1 illustrates four separate insertion points, it is also taught that fewer than four can be used, such as two ventrally and one dorsally. The present invention further teaches providing adjustable shoulder harness by including rigid loops 55 at the insertion point below the slide 45 included on the shoulder harness. Again, two loops may be placed ventrally, or a single loop placed dorsally in a configuration which allows the wearer to maximize the comfort of the shoulder harness 40. The present invention also teaches coupling the shoulder harness 40 to the belt 10 using hook and pile to provide maximum adjustability and maximize the comfort of the shoulder harness 40.

Also illustrated in FIG. 1 is a garment rear attachment surface 80 positioned on the interior surface of the first adjustable compression belt 10. The garment rear attachment 80 is an attachment mechanism such hook and loop or other means, and provides for the support to releasably and selectively couple to a garment.

In addition to the above-discussed shoulder harness 40, the present invention teaches an adjustable lower anchor or pelvic harness or second vertical movement anchor 60 constructed so as to prevent the lumbar support from sliding too high on the user. The second anchor 60 has a number of embodiments. In one embodiment, second anchor 60 comprises a buckle receiver 65 coupled to the external surface of the second adjustable compression belt 30. FIG. 1. Thus, the second adjustable compression belt 30 allows the user not only to manipulate the amount of compression exerted by the belt on the back of the wearer, but additionally allows the wearer to adjust the position of the pelvic harness 60 and thus maximize both the security and comfort of the lumbar support.

To further facilitate both the positioning and the comfort of the lumbar support, buckle receiver 65 and buckle 70 have a pelvic harness slide adjustment 75 positioned so that tension along the pelvic harness can be increased or decreased depending on the activity as well as the anatomy of the wearer. Second anchor 60 controls movement of support 5 to prevent undesirable hiking or climbing of support 5 up the torso of the user.

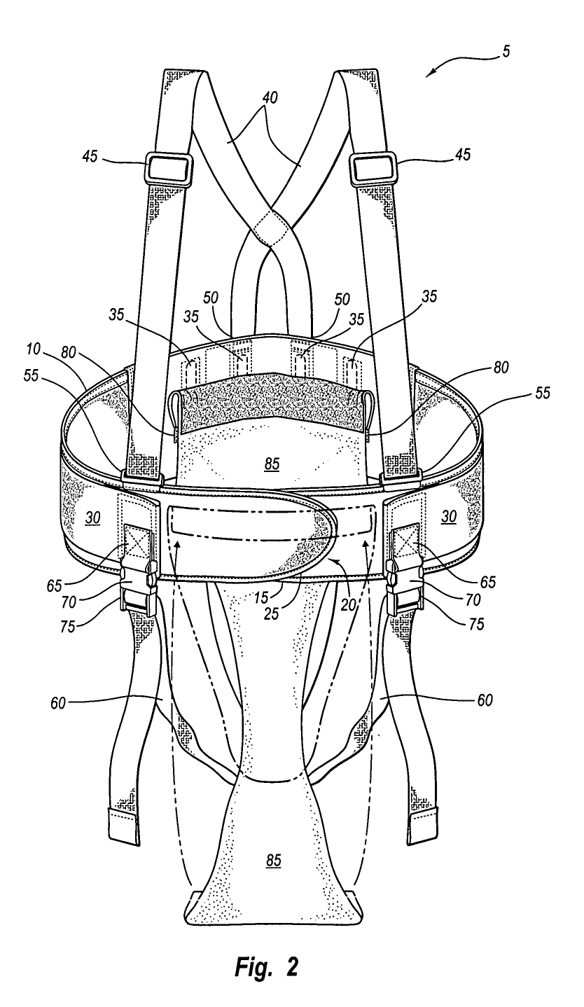

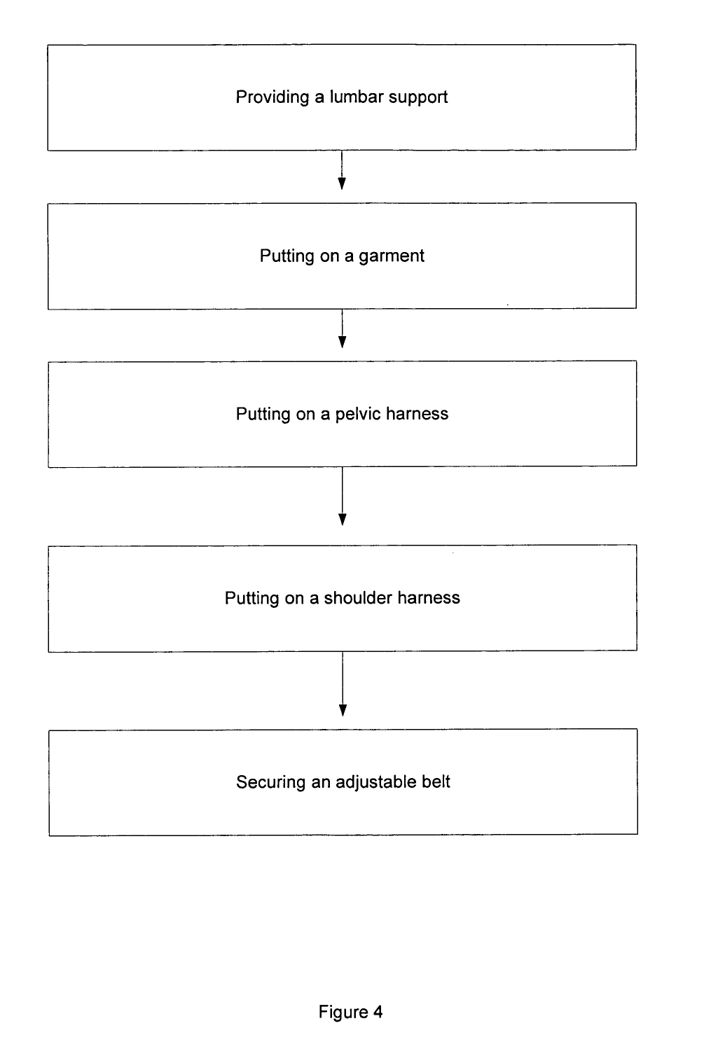

Referring now to FIG. 2 wherein an alternative embodiment of the lumbar support 5 is illustrated. In addition to the features described previously, a harness 60 is illustrated. The harness shown in FIG. 2 will fit about the crotch of the wearer to increase the surface area of the harness, and thus reduce the pressure the harness exerts at any one point on the wearer. In addition to the present harness of FIG. 2, shorts or pants are also contemplated by the present invention. Indeed the present invention teaches a lumbar support with no identifiable harness attached thereto, however, there may also be selectively and releasably coupleable shorts, pants, or other under or outer garment worn by the user coupled to support 5 of FIG. 4. In any event, like harness 60 of FIG. 2, pants of FIG. 4 anchor support 5 to prevent undesirable hiking or climbing of support 5 up the torso of the user. Lower anchor 60 also prevents twisting of the support 5 by providing additional anchor area.

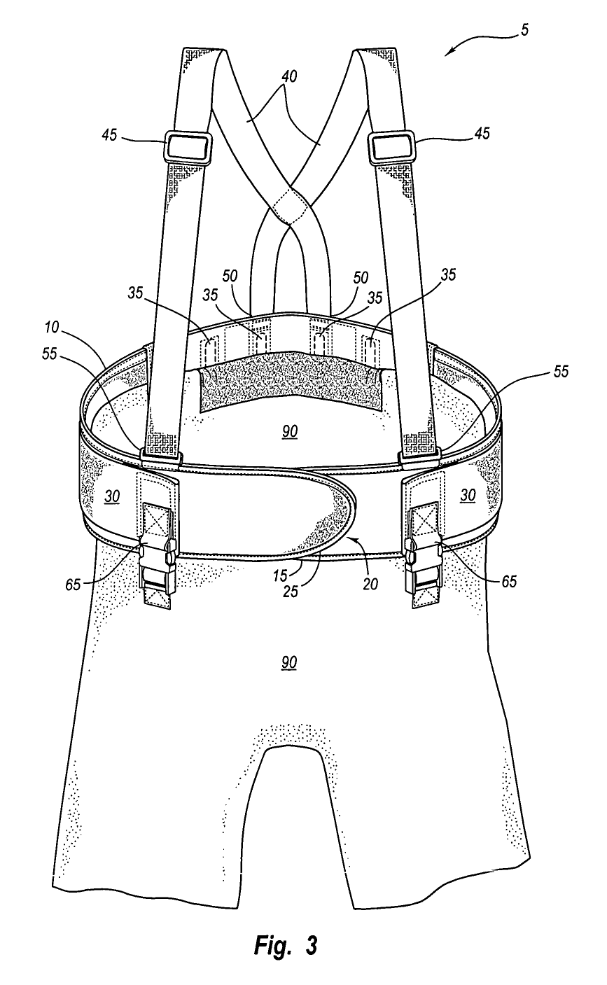

With reference to FIG. 3, there is illustrated an alternative embodiment of the present invention wherein the lower anchor is integrated into the garment 60 thus eliminating any straps as disclosed previously. While FIG. 3 illustrates that garment 60 is coupled to belt 30, the embodiments taught by the present invention contemplates a releasably coupleable garment 60 which would be indistinguishable from standard shorts or pants. The garment 60 as taught by the present invention may be configured to have a coupling means attached thereto to enable the garment to releaseably couple a buckle to the belt 30. The garment 60 may be insertable in that the garment 60 may be worn independent of the belt 30 and vice versa. The coupling means may also be a hook and pile system for coupling the contacting surfaces of belt 30 with garment 60. The coupling means may also be related from a zipper, hook and eye, snaps, clasps, buckles and any other coupling means known in the fastening art.

Referring now to FIG. 4 which illustrates a method of using the lumbar support. A user may utilize any of these steps in any order, and it is the result which is the method claimed. As taught by the present invention, one embodiment of the method comprises providing an adjustable lumbar support 305 having a pelvic harness below to an adjustable belt, and a shoulder harness above to the adjustable belt. Also taught by the adjustable lumbar support is a releasably and selectively coupleable garment. The method teaches putting on the garment 310, and then putting on the pelvic harness 315. However, the simultaneous with the garment 301 where the garment and harness are coupled. The pelvic harness provides an anchor below to the adjustable belt. The method next teaches putting on a shoulder harness 320 over the user's shoulders thus providing a counter anchor to the pelvic harness 315. As taught by the present invention, anchor points below and above to the adjustable belt allows the wearer to secure the adjustable belt in a specific region, the lumbar region and not have to continually manipulate the belt's position. Indeed the final step taught by the present method is securing the adjustable belt 325 in the correct position, thus increasing the comfort, safety and support of the adjustable belt to prevent vertical movement or lateral twisting of support 5 on or about the user.

What is claimed:

1. An apparatus for providing lumbar support of a user, the support comprising:
an adjustable belt;
a shoulder harness attached to and extending above the adjustable belt; and
a pelvic harness attached to and extending below the adjustable belt, whereby the shoulder harness and the pelvic harness minimize vertical or lateral movement of the support about the user.

2. The adjustable belt of claim 1 wherein the adjustable belt has at least five adjustment points.

3. The adjustable belt of claim 1 wherein the adjustable belt has at least one constricting and at least one relaxing adjustment mechanism.

4. The adjustable belt of claim 1 wherein a person wearing the belt may draw the belt above, below, and medially.

5. The apparatus of claim 1 wherein the shoulder harness of claim I provides an anchor above to the belt.

6. The pelvic harness of claim 1 wherein the pelvic harness creates an anchor below to the adjustable belt.

7. The apparatus of claim 1 wherein the pelvic harness is selectively adjustable.

8. The pelvic harness of claim 1 further comprising a selectively releasable garment.

9. A method of securing a support in the lumbar region of the abdomen comprising:
providing a support comprising an adjustable belt having a shoulder harness attached to the adjustable belt and a pelvic harness attached to the adjustable belt;
donning the pelvic harness;
donning the shoulder harness;
securing the adjustable belt; and
adjusting the tension of the shoulder harness and the pelvic harness so as position and secure the adjustable belt in the lumbar region.

10. The method of claim 9 further comprising adjusting the constrictiveness of the adjustable belt by tightening or loosening the adjustable belt.

11. The method of claim 9 further comprising reducing the pressure on the pelvic harness straps by inserting a releasably coupleable garment between said harness and said belt.

12. The method of claim 9 further comprising adjusting the position of the lumbar support by adjusting the position of the garment.

13. The method of claim 9 further comprising adjusting the position of the lumbar support by adjusting the tension of the shoulder harness.

14. A system for supporting a lumbar region comprising an adjustable belt having a first vertical movement harness and a second vertical movement harness coupled to the adjustable belt.

15. The system of claim 14 further comprising a selectively insertable pelvic harness garment.

16. The system of claim 14 wherein the user adjusts the length of the first harness and the second harness to secure the vertical position of the lumbar support
